(19) United States
(12) Patent Application Publication
(10) Pub. No.: US 2005/0086708 A1
(43) Pub. Date: Apr. 21, 2005

(54) Animal models of retinal tumorigenesis

(75) Inventor: Michael Allen Dyer, Memphis, TN (US)

Correspondence Address: ST. JUDE CHILDREN'S RESEARCH HOSPITAL;OFFICE OF TECHNOLOGY LICENSING, 332 N. LAUDERDALE, MEMPHIS, TN, 38105, US

(73) Assignee: St. Jude Children's Research Hospital

(21) Appl. No.: 10/962,832

(22) Filed: Oct. 12, 2004

Related U.S. Application Data
(60) Provisional application No. 60/512,803, filed on Oct. 20, 2003.

(51) Int. Cl. A01K 67/027

(52) U.S. Cl. 800/10; 800/21

(57) ABSTRACT

Complimentary animal models for retinoblastoma which recapitulate conditions found in the eye of human retinoblastoma patients in an animal are provided. These models are generated by introducing an agent capable of giving rise to a retinoblastoma into the developing eye of an immunologically naive animal. In one model the agent comprises cells which are capable of giving rise to a retinoblastoma. In another model the agent comprises a vector capable of expressing an oncogene which, when expressed in a transfected cell, can give rise to a cell mass that mimics the early stages of retinoblastoma formation. These models can be used to study retinoblastoma and screen for, or characterize, inhibitory agents. These models may also be used to study the influence of genotype or engineered genes or gene deficiencies (knock-outs) on the development of retinoblastoma.

### CROSS REFERENCE TO RELATED APPLICATIONS

This application claims priority under 35 U.S.C. § 119(e) to U.S. Provisional Application Ser. No. 60/512,803 filed Oct. 20, 2003, which is incorporated by reference herein in its entirety.

### FIELD OF THE INVENTION

This invention relates to methods for studying cancer generally and retinal tumors specifically, particularly retinoblastoma.

### BACKGROUND

Retinoblastoma

Multipotent retinal progenitor cells give rise to the seven different classes of retinal cell types in an evolutionarily conserved birthorder. Livesey, F. J., and Cepko, C. L., Nat Rev Neurosci, 2:109-118 (2001). Ganglion cells are among the first retinal cells to be produced, while bipolar cells are among the last. Research over the past several years has led to a competence model for retinal cell fate specification. Cepko, C. L. et al., Proc Natl Acad Sci USA, 93: 589-95. (1996). Retinal progenitor cells are believed to progress in a unidirectional manner through distinct stages of competence, which are defined by the ability to generate subsets of retinal cell types.

The coordination of cell cycle exit with cell fate specification during retinal development is important for determining the size and composition of the mature retina. In humans, if retinal progenitor cells fail to exit the cell cycle during development, a malignant retinoblastoma may form. Retinoblastoma is a neoplastic condition of the retinal cells, observed almost exclusively in children between the ages of 0 and 7 years. It affects between 1 in 34,000 and 1 in 15,000 live births in the United States. L. E. Zimmerman, “Retinoblastoma and retinocytoma”, In W. H. Spencer (ed.), Ophthalmic Pathology: an Atlas and Textbook, Vol. II, Philadelphia: W. B. Saunders Co., pp. 1292-1351 (1985). Retinoblastomas are characterized by small round cells with deeply stained nuclei, and elongated cells forming rosettes. They usually cause death by local invasion, especially along the optic nerves. If untreated, the malignant neoplastic retinal cells in the intraocular tumor travel to other parts-of the body, forming foci of uncontrolled growth which are always fatal.

The current treatment for a retinoblastoma is enucleation of the affected eye if the intraocular tumor is large. For small intraocular tumors radiation therapy, laser therapy, or cryotherapy is preferred. There is no known successful treatment for metastatic retinoblastoma.

In 30-40% of cases of retinoblastoma, the affected individual carries a heritable predisposition to retinoblastoma and can transmit this predisposition to his or her offspring as a dominant trait (A. G. Knudson, “Mutation and cancer: Statistical study of retinoblastoma”, Proc. Natl. Acad. Sci., 68: 820-23 (1971)). Carriers of this retinoblastoma-predisposing trait are at a greatly elevated risk for development of several other forms of primary cancer, notably osteosarcoma and soft-tissue sarcoma.

The genetic locus associated with familial retinoblastoma has been assigned to the q14 band of human chromosome 13 (R. S. Sparkes et al., Science, 208: 1042-44 (1980)). The gene that is mutated in familial retinoblastoma has been identified and is called the retinoblastoma susceptibility gene (RB). It is widely accepted that when a defective copy of this gene is passed along to a child from their parent, the second allele is mutated during DNA replication in retinal progenitor cells during development in utero. Children who have had one eye affected by retinoblastoma or who are related to someone with retinoblastoma may be genetically predisposed and therefore at risk of developing the disease. These individuals routinely are tested for retinoblastoma every 2-3 months by an ocular examination procedure which requires placing the child under general anesthesia. The inactivation of both copies of the RB gene is the initiating event in all retinoblastoma tumors. Thus, children without a family history can also develop retinoblastoma by acquiring mutations in their RB gene. However, in these cases, both copies of the gene must be mutated during development and as a consequence, the number of tumors that form in their eyes is dramatically reduced.

The Retinoblastoma (Rb) Gene Family

The Rb gene family comprises three members, Rb, p107 and p130, which lie at the center of the regulatory network that controls cell cycle exit (Sears, R. C., and Nevins, J. R., J Biol Chem, 277: 11617-20. (2002); Ferguson, K. L., and Slack, R. S., Neuroreport, 12: A55-62. (2001)). Following mitosis, Rb family members associate with E2F transcriptional regulators bound to their cognate DNA sequences upstream of genes important for cell cycle progression, apoptosis, or differentiation. Expression of E2F regulated promoters may be upregulated or silenced by Rb family members through chromatin remodeling or interactions with transcriptional regulators. If a cell is going to proceed through another round of cell division, distinct cyclin/cyclin dependent kinase (CDK) complexes serially phosphorylate the Rb family members (Harbour, J. W. et al., Cell, 98: 859-69. (1999); Zhu, L. et al., Embo J, 14: 19041(1995);Ewen, M. E. et al., Science, 255: 85-7 (1992); Faha, B. et al, Science, 255: 87-90. (1992); Castano, E. et al, Mol Cell Bio., 18: 5380-91. (1998); Dick, F. A. et al., Mol Cell Biol, 20: 3715-27. (2000); Dick, F. A., and Dyson, N. J., J Virol, 76: 6224-34. (2002); Meloni, A. R. et al., Proc Natl Acad Sci USA 96: 9574-9. (1999); Woo, M. S. et al., Mol Cell Biol, 17: 3566-79. (1997); Smith, E. J., and Nevins, J. R., Mol Cell Biol. 15: 338-44. (1995)). These phosphorylation events interfere with the ability of Rb, p107 and p130 to regulate transcription at E2F responsive promoters. Alternately, if a cell is going to exit the cell cycle and undergo terminal differentiation, phosphorylation of Rb family members is blocked.

Several unique properties of Rb family members have been described. First, Rb, p107 and p130 are differentially expressed during development (Jiang, Z. et al., Oncogene, 14: 1789-97. (1997)) and they are expressed during different phases of the cell cycle (reviewed in Classon, M., and Dyson, N., Exp Cell Res. 264: 135-47 (2001)).

Second, Rb family members exhibit preferential binding to distinct E2F family members (reviewed in Nevins, J. R., Hum Mol Genet. 10: 699-703. (2001); Nevins, J. R., Cell Growth Differ. 9: 585-93. (1998);Dyson, N., Genes Dev. 12: 2245-62. (1998)).

Third, p107 and p130 form stable complexes with cyclinA/CDK2 and cyclin E/CDK2 (Ewen, M. E. et al., Science, 255: 85-7 (1992); Faha, B. et al., Science, 255, 87-90 (1992);Lees, E. et al., Genes Dev. 6: 1874-85. (1992); Morris, E. J., and Dyson, N. J., Adv Cancer Res. 82: 1-54 (2001) whereas Rb does not form such complexes.

Fourth, Rb has been found to associate with more than 110 different proteins (Morris, E. J., and Dyson, N. J., supra (2001)), some of which represent transcription factors that are important for retinal development (Chx10, Pax6). Most of these Rb-binding proteins have not yet been tested for their ability to bind to p107 or p130 but there are a handful of examples of specific interactions between transcription factors and individual Rb family members (reviewed in Morris, E. J., and Dyson, N. J., supra (2001)). For example, Pax5 binds to Rb and p107 but not to p130 (Eberhard, D., and Busslinger, M., Cancer Res.: 59 (1999)).

Taken together, these observations suggest that different Rb family members may initiate or modulate developmental programs in distinct tissues during development. Tissue-specific developmental defects observed in mice carrying targeted deletions of individual Rb family members or E2F genes support this idea (Gaubatz, S. et al., Mol Cell. 6: 729-35. (2000); Humbert, P. O. et al., Mol Cel. 6: 281-91 (2000); Lee, E. Y. et al., Genes Dev. 8: 2008-21. (1994); Slack, R. S., and Miller, F. D., Dev Gene., 18: 81-91 (1996); Rempel, R. E. et al., Mol Cell. 6: 293-306. (2000); Lindeman, G. J. et al., Genes Dev. 12: 1092-8. (1998)).

Rb was the first tumor suppressor identified in humans (Friend, S. H. et al., Nature 323: 643-646 (1986); Lee, W. H. et al., Science 235: 1394-1399 (1987)). Inheritance of a defective allele of RB results in an increased susceptibility to retinal tumors through inactivation of the normal allele during mitotic cell division (reviewed in DiCiommo, D. et al., Semin Cancer Biol. 10: 255-69 (2000)).

Since this pioneering work on retinal tumors, the Rb gene or the Rb pathway has been found to be disrupted in a wide range of cancer cell types (Nevins, J. R., Hum Mol Genet. 10: 699-703. (2001); Weinberg, R. A., Cell 81: 323-30 (1995); Sherr, C. J., Science 274: 1672-7. (1996)). There is evidence indicating that p107 and p130 can also act as tumor suppressors(Masciullo, V. et al., Int J Oncol 17: 897-902. (2000); Ginsberg, D. et al., Genes Dev 8: 2665-79. (1994); Beijersbergen, R. L. et al., Genes Dev 8: 2680-90 (1994); Zalvide, J., and DeCaprio, J. A., Mol Cell Biol 15: 5800-10 (1995); Hahn, W. C., and Weinberg, R. A., Nat Rev Cancer 2: 331-41 (2002); Christensen, J. B., and Imperiale, M. J., J Virol 69: 3945-8. (1995); Slebos, R. J. et al., Proc Natl Acad Sci US A 91: 5320-4. (1994); Sage, J. et al., Genes Dev 14: 3037-50 (2000)).

While retinoblastoma results from inactivation of the RB gene, other tumors carry mutations in different cell cycle components. For example, pancreatic tumors often harbor mutations in the p16 cyclin kinase inhibitor (Sherr, C. J., Science 274: 1672-7 (1996); Sherr, C. J., Cancer Res 60: 3689-95 (2000)). This may reflect the tissue-specific utilization of the different cell cycle components or unique compensatory or redundant mechanisms in different tissues.

Mice with engineered deficiencies in Rb family members have been generated. Maandag, E. C. et al., Embo J 13: 4260-4269 (1994); Lee, M. H. et al., Genes Develop. 10: 1621-1632 (1996). However, these mice do not accurately recapitulate the development of retinoblastoma that occurs in humans with corresponding genetic deficiencies. This is partly because the expression of Rb family members and their role during development differs significantly between mice and humans. Second, it appears that reciprocal compensation occurs in the mouse retina between Rb and p107 such that p107 expression and activity is altered in mice deficient in Rb activity to compensate for this deficiency and vice versa. This compensation mechanism doe not appear to occur in humans.

### SUMMARY OF THE INVENTION

Models for retinoblastoma which recapitulate conditions found in the eye of human retinoblastoma patients in an animal are provided. These models are generated by introducing an agent capable of giving rise to a retinoblastoma into the developing eye of a newborn, immunologically naive animal. In one model the agent comprises cells which are capable of giving rise to a retinoblastoma. In another model the agent comprises a vector capable of expressing an oncogene which, when expressed in a transfected cell, can give rise to a retinoblastoma. These models can be used to test various agents and therapeutic regimens for activity against retinoblastoma and other cancers having susceptibilities in common with retinoblastoma.

Cells capable of giving rise to a retinoblastoma may be derived from an established cell culture. These cells may also be derived from excised tissue such as a tissue excised from a retinoblastoma tumor of particular interest. These cells include, but are not necessarily limited to, retinoblastoma cells, retinal progenitor cells and retinal stem cells. These cells may be engineered to express, or not express, a gene of interest before introduction into the eye of the host animal.

Oncogenes which may be used to induce retinoblastoma formation in transfected cells of the developing eye of the host animal include, but are not necessarily limited to, viral oncogenes such as E1A, E6, E7 and Tag and cellular oncogenes such as Ras, Myc, Abl and Erk.

Any animal which is receptive to the growth and proliferation of a retinoblasotoma from introduced cells in the developing eye can be used in these models. A standard laboratory research animal, such as a mouse, rat, rabbit or monkey is preferred for use in these models. The animal model of retinoblastoma generated through the method taught herein represents another aspect of the invention.

Animal models of retinoblastoma prepared according to the methods of the invention can be used to screen and characterize drugs for activity against retinoblastoma, as well as to study the biology and development of retinoblastoma.

### DETAILED DESCRIPTION OF THE INVENTION

Definitions:

Immunologically naive: A premature stage of the immune system when T-cells do not yet distinguish between cells from their own body and foreign cells. Mammals are born immunoligically naïve.

retinal progenitor cell: Retinal progenitor cells are immature cells in the retina that are dividing and capable of giving rise to retinal neurons and glia. In addition, retinal progenitor cells have the potential to give rise to retinoblastoma following genetic alterations. These cells can be identified from a host based on their proliferation properties and their ability to give rise to neurons and glial cells in vitro. retinal stem cell: Retinal stem cells are the same as retinal progenitor cells except thay they have the unique property to self-renew leading to immortality. These cells can be isolated from a host and distinguished from retinal stem cells by their ability to self-renew in culture.

retinoblastoma cell: An undifferentiated or de-differentiated cell derived from a retinoblastoma tumor. Retinoblastoma cells can propagate and give rise to further retinoblastoma tumors.

### DESCRIPTION OF THE INVENTION

The present invention fills a need for an animal research model of retinoblastoma. The models provided can be used to study retinoblastoma in the context of a living nonhuman organism. These models can also be used to screen for useful therapeutic agents that can inhibit retinoblastoma or characterize the effects of previously identified inhibitory agents in the context of a living animal.

Both retinoblastoma models of the present invention are prepared from two basic components: (1) the host animal, and (2) an agent capable of giving rise to retionoblastoma when introduced into the eye of the host animal. In the first model described below this agent comprises cells capable of giving rise to retinoblastoma. In the second model described below this agent comprises a vector containing an oncogene which, when expressed in a transfected cell, can give rise to a retinoblastoma or a mass of proliferating cells which mimics the early stages of cellular proliferation from a retinoblastoma cell.

The Host Animal

Any animal which is receptive to the introduction and growth of human retinoblastoma cells in the eye can be used as the host for these models. Common laboratory animals which are well characterized and whose handling is familiar to researchers are preferred for use as host animals. This includes, but is not limited to, a monkey, a mouse, a rat and a rabbit. Each of these animals has its own peculiar attributes well known to those of skill in the art which can be taken into account when choosing an appropriate model for any given purpose. Attributes of such animals which are of particular significance for purposes of their use in the present invention include, but are not limited to, the size, accessibility and manipulability of the eye, the genetic relatedness of the animal to humans, and the length of time that the animal remains immunologically naive during development and the stage of eye development during the period of immunological incompetence.

Regardless of the species chosen, another attribute of the host animal to be considered is its genotype with respect to its susceptibility to retinoblastoma growth and metastasis. In certain circumstances it may be desirable to use a host animal that has a genetic predisposition to the occurrence of retinoblastoma specifically or to cancers generally. Such a host animal may have one or more genetic defects associated specifically with retinoblastoma, such as defects in Rb gene family members Rb, p107 and/or p130. Alternatively, the host animal may have one or more genetic defects associated with cancer generally, such as defects in the p53 or p19ARF genes (Quelle, D. E. et al., “Alternative reading frames of the INK4a tumor suppressor gene encode two unrelated proteins capable of inducing cell cycle arrest”, Cell 83: 993-1000 (1995).

For the second model described below, the genotype used can affect the progression of retinoblastoma in the host animal. If a normal animal with no genetic susceptibility to cancer formation is used in this model, cells in which the oncogene is effectively inserted and expressed will tend to proliferate into a cell mass similar to the early stages of retinoblastoma formation from a clonal foci, but will not progress further. If an animal that is susceptible to cancer formation is used, particularly an animal that has a defect in p53 such as a p53 knock-out, cells in which the oncogene is effectively inserted and expressed will proliferate and continue to progress into retinoblastoma.

As another alternative, the host animal used may have a defined genotype with an uncertain effect on retinoblastoma formation. In this context the animal host is used to study the effect of its particular genotype on retinoblastoma formation (see “Utility” below).

The First Animal Model

To generate the first model, cells capable of giving rise to retinoblastoma are introduced into the host animal at a time when the animal is immunologically naive to avoid immune rejection of the introduced cells and/or the use of immunosuppressive drugs. For most animals, including mice, rats, rabbits and monkeys, this immunologically naive state naturally exists in utero and persists for a period of a few hours to a few days after birth.

Another host animal consideration when it comes to the introduction of cells is the developmental stage of the eye. Optimally introduction will occur at a developmental stage corresponding to the in utero developmental stage in which retinoblastoma normally forms in humans. This human developmental stage is contemplated to occur in mice and rats immediately after birth at a time when these animals remain immunologically naive. Therefore, when a mouse or rat is used as the host animal, the cells capable of giving rise to retinoblastoma are preferably introduced sometime soon after birth and preferably immediately after birth to best mimic the development of retinoblastoma in humans. See Clancy, B., Darlington, R. B., and Finlay, B. L., “Translating developmental time across mammalian species”, Neuroscience 105(1): 7-17 (2001).

Any cell type capable of giving rise to retinoblastoma can be used in the method taught herein to generate a retinoblastoma in the host animal. This includes, but is not necessarily limited to, retinoblastoma cells, retinal progenitor cells and retinal stem cells. Retinoblastoma cells are the preferred cell type for this purpose.

Human cells can be introduced into the host animal to generate the retinoblastoma model of the invention. Alternatively, cells originating from the host animal species which are capable of giving rise to retinoblastoma may also be used. Use of cells from the same species as the host animal has the advantage of minimizing the risk of host rejection when the cells are introduced or thereafter. However, such a model may not mimic human retinoblastoma as closely as a model in which the retinoblastoma originates from human cells.

Cells capable of giving rise to retinoblastoma used in the method of the invention can be derived either from an established cell culture, a new primary cell culture, or from freshly excised tissue, particularly retinoblastoma tissue. Established human retinoblastoma cell cultures useful in the present method include, but are not limited to, Y79 (ATCC deposit no. HTB-18) and Weril (ATCC deposit no. HTB-169). See also Reid, T. W. et al., J Natl Cancer Inst. 53: 347-360 (1974); McFall, R. C. et al., Cancer Res. 37: 1003-10 (1977); Griegel, S. et al., Differentiation, 45: 250-257(1990). Established cell cultures can also be made by continued culturing of primary cell cultures under appropriate conditions. It has been found that culturing in RPMI medium supplemented with 10% fetal calf serum and L-glutamine is sufficient for the growth of many primary retinal tumors in approximately 2 weeks. See Greigel, S. et al., “Newly established human retinoblastoma cell lines exhibit an “immortalized” but not an invasive phenotype in vitro”, Int. J Cancer 46: 125-132 (1990). Growth on human fibroblast feeder layers, or extracellular matrix components was not necessary for growth of retinoblastoma in those studies.

New primary cell cultures can be made by excising tissue containing the cells of interest (approximate 0.5 mm cube of tumor), placing the tissue in appropriate culture medium (RPMI-10% fetal calf serum) under sterile conditions and monitoring the growth of cells in the medium. Griegel, S., et al., Id. (1990).

Cells capable of giving rise to retinoblastoma may also be obtained through excision of appropriate tissue from a human or animal. Excised retinoblastoma tumor tissue is preferred, although excised tissue comprising retinal progenitor and /or retinal stem cells may also be used. For the preparation of an animal retinoblastoma model that is most similar to a specific patient suffering from retinoblastoma, retinoblastoma tumor tissue is excised from the patient and used to inoculate the animal host. The model derived from such retinoblastoma tissue will most closely mimic the retinoblastoma of the patient from which the tissue was derived. Such a model could be used to screen for inhibitory agents which are effective against a specific tumor, or to compare the efficacy of available inhibitory agents to choose which agent would be expected to work best against a specific tumor.

Cells capable of giving rise to retinoblastoma used in the method of the invention can be engineered to express a gene of interest or to render the cell deficient in the expression of a gene of interest using standard techniques. See, e.g. Fukuda, K. et al., “Application of efficient and specific gene transfer systems and organ culture techniques for the elucidation of mechanisms of epithelial-mesenchymal interaction in the developing gut”, Dev Growth Differ. 42(3): 207-11 (2000). If desired, the gene of interest can be engineered to be expressed constitutively in the cell, or to be expressed in a regulated or inducible fashion. Genes of interest that one may wish to engineer for expression or nonexpression in these cells prior to introduction in the model particularly include the Rb gene family members Rb, p107 and p130 and cell cycle proteins that are important for retinal development. Dyer, M. A. and C. L. Cepko, Nat. Rev. Neurosci. 2(5): 333-42 (2001). Genes of interest that one may wish to engineer for expression in these cells also include marker genes such as green fourescent protein, beta-galactosidase, alkaline phosphatase and selectable markers such as cell cycle genes, genes that induce differentiation or regulate proliferation such as Prox1, Six3 or Chx10 (Dyer, M A., Cell Cycle 2(4): 350-357 (2003)) and genes involved in apoptosis and angiogenesis.

The amount of cells introduced into the host animal is not critical as long as it is sufficient to cause the growth of a retinoblastoma tumor. This amount is preferably within the range of about fifty-(50) to ten thousand (10,000) cells and most preferably within the range of about five hundred (500) to one thousand (1000) cells. These cells can be introduced into the eye of the host animal with no need for any special preparation using conventional sterile techniques. Care is taken to avoid damaging the lens and the retina. Injections are carried out exactly as with in vivo retroviral injections as described in Turner, D. L. and Cepko, C. L. A., “Common progenitor for neurons and glia persists in rat retina late in development”, Nature 328(6126):131-6 (1987), except that the cells are targeted to the vitreous of the eye.

A host animal can be used as a retinoblastoma model immediately after the cells capable of giving rise to retinoblastoma have been introduced in its eye(s). One may choose to introduce cells into only one eye and use the other as a control. The introduced cells tend to give rise to a retinoblastoma tumor that completely fills the eye in about 15 to about 30 days. In those cases where the cells have been engineered to express a marker, such as green fluorescent protein, growth of the tumor from the introduced cells can be monitored through detectable expression of the marker.

The Second Animal Model

To generate the second model, a vector capable of expressing an oncogene is used. Preferably, a retroviral vector is used, more preferably the retroviral vector is replication incompetent. See Cepko, C., “Transduction of genes using retroviral vectors”, Current Protocols in Molecular Biology, ed. By Ausubel, F. M. et al., pub. By John Wiley and Sons, New York (1997); Cepko, C. L. et al., “Lineage analysis using retroviral vectors”, Current Topics in Developmental Biology 36: 51-74 (1998); Cepko, C. L. et al., “Construction and applications of a highly transmissible murine retrovirus shuttle vector”, Cell 37:1953-1062 (1984); Cepko, C. L. et al., “Lineage analysis with retroviral vectors”, Methods Enzymol. 327: 118-145 (2000). Other vector types that can be used include lentiviruses and adenoviruses. See Cockrell, A. S. & Kafri, T., “HIV-1 vectors: fulfillment of expectations, further advancements, and still a way to go”, Curr HIV Res 1: 419-39 (2003); Saukkonen, K. & Hemminki, A., “Tissue-specific promoters for cancer gene therapy”, Expert Opin Biol Ther 4: 683-96 (2004).

The oncogene used may be any oncogene that can give rise to retinoblastoma when expressed in a cell. This includes, but is not necessarily limited to viral oncogenes such as E1A (See Frisch, S. M. & Mymryk, J. S., “Adenovirus-5 E1A: paradox and paradigm”, Nat Rev Mol Cell Biol 3: 441-52. (2002)) and Tag (See Dean, F. B. et al., “Simian virus 40 (SV40) DNA replication: SV40 large T antigen unwinds DNA containing the SV40 origin of replication”, Proc Natl Acad Sci U S A 84: 16-20 (1987)) and cellular oncogenes such as Ras (See Coleman, M. L. et al., “RAS and RHO GTPases in G1-phase cell-cycle regulation”, Nat Rev Mol Cell Biol 5:355-66 (2004)), Myc (See Setoguchi, M. et al., “Insertional activation of N-myc by endogenous Moloney-like murine retrovirus sequences in macrophage cell lines derived from myeloma cell line-macrophage hybrids”, Mol Cell Biol 9: 4515-22 (1989)), Abl (See Witte, O. N., “Functions of the abl oncogene”, Cancer Surv 5: 183-97 (1986)) and Erk (See Crews, C. M. et al., “Mouse Erk-1 gene product is a serine/threonine protein kinase that has the potential to phosphorylate tyrosine”, Proc NatlAcad Sci USA 88: 8845-9 (1991)). See also White, R. J., “RNA polymerase III transcription-a battleground for tumour suppressors and oncogenes”, Eur. J. Cancer 40: 21-27 (2004); Felsher, D. W., “Cancer revoked: oncogenes as therapeutic targets”, Nature Reviews 3: 375-380 (May 2003). Preferably the E1A viral oncogene is used.

In this model, the vector is introduced into the eye of the host animal. It is preferable to use a host animal at the same immunologically naïve developmental stage as in the first model. However, in this model host animals at other earlier or later developmental stages can be used. A small hole is made in the cornea at the corneal-scleral boundary using a 30 gauge (Ga) needle. Then a blunt 33 Ga needle attached to a 5 microliter Hamilton syringe is inserted into the eye through this hole. The needle is inserted through the retina to the subretinal space and 0.5 microliters of vector is delivered.

The amount of vector introduced into the eye can vary, but optimally the concentration of vector used will be sufficient to lead to 1-5 clonal transformation events in each treated eye. This level of transformation events most closely mimics the typical development of human retinoblastoma from isolated mutational events in single cells. The concentration of vector needed to induce 1-5 transformation events in a treated eye can be determined by titering the virus on cultured mouse fibroblasts such as NIH 3T3 cells. See Cepko, C., “Transduction of genes using retroviral vectors”, in Current Protocols in Molecular Biology (eds. Ausubel, F. M. et al.) (John Wiley and Sons, New York, 1997). Briefly, the virus is serially diluted and added to dividing mouse fibroblasts. Two days later, the cells are fixed with paraformaldehyde and stained for alkaline phosphatase expression or the specific reporter gene in the retrovirus. The number of infected clones of cells is then multiplied by the dilution factor and this is the titer in infectious units per ml. The inventor has found that, in this system, 1x10⁶ infectious units per ml is sufficient to induce 1-5 clonal tumors in vivo when administered by subretinal injection (0.5 microliters).

In this model, the genotype of the host animal used can affect the progression of retinoblastoma. If a normal animal with no genetic susceptibility to cancer formation is used in this model, cells in which the oncogene is effectively inserted and expressed will tend to proliferate into a cell mass similar to the early stages of retinoblastoma formation from a clonal foci, but will not progress further. If an animal that is susceptible to cancer formation is used, particularly an animal that has a defect in p53 such as a p53 knock-out, cells in which the oncogene is effectively inserted and expressed will proliferate and continue to progress into retinoblastoma.

Utility

The animal models produced according the methods taught herein can be used to screen for or characterize inhibitors of retinoblastoma and other cancers with common susceptibilities. Candidate inhibitors can be introduced into the animal model by any desired means including, but not limited to, oral ingestion, intravenous injection, injection in the eye, and intraperitoneal injection. These candidate inhibitors may be introduced into the animal before introduction of the cells capable of giving rise to retinoblastoma, during this introduction (either separately or by co-administration), or thereafter as long as the animal remains viable and alive. The effect of the candidate inhibitor can be determined by monitoring and/or measuring its effect upon growth of the introduced cells and development of a retinoblastoma tumor.

One may also use the method of the invention to study the influence of genotype on retinoblastoma growth and metastasis. This would simply require using animals with the genotype(s) of interest as the host animal in the method taught herein. For example, knockout mice with defects in angiogenesis or glial cell activation could be used to study the effect of these defects on retinoblastoma growth and metastasis. Retinoblastoma could be introduced into animals with a variety of genotypes using the methods taught herein to compare the relative susceptibility of each genotype to retinoblastoma. This process could also be used to screen for genotypes which confer resistance or reduced susceptibility to retinoblastoma or to test genetically engineered animals for such resistance or reduced susceptibility.

### EXAMPLES

### Example 1

### Mouse Models for Retinoblastoma

### SUMMARY

Targeted cancer therapies rely on a thorough understanding of the signaling cascades, genetic changes, and the compensatory programs that are activated during tumorigenesis for each tumor cell type. Increasingly, pathologists are called upon to interpret molecular profiles of tumor specimens in order to target new therapies. In many cases, this can be a challenge because cancer is a heterogeneous disease. Not only do tumors change over time in individual patients, but also the genetic lesions that lead from a preneoplastic lesion to malignant transformation can differ substantially from patient to patient. For childhood tumors of the nervous system, the challenge is even greater because tumors arise from progenitor cells in a developmental context that is entirely different from that of the adult tissue. Even more important, the cells of origin, neural progenitor cells, normally exhibit considerable temporal and spatial heterogeneity during development. Thus, not only do we need to understand the individual steps leading from a preneoplastic lesion to metastatic cancer but also we need to understand the underlying mechanisms regulating normal development of the nervous system. Many of the most important advances in our understanding of nervous system development and tumorigenesis have come from model genetic systems such as the mouse. This example highlights a mouse model of childhood retinoblastoma. Emphasis is placed on how our understanding of the normal developmental processes combined with this mouse model and the molecular pathology of the human diseases can provide the information needed to target cancer therapy with efficacy.

Introduction

In many forms of adult cancer, the cell of origin is well understood based on the location of the tumor, the molecular markers expressed in the tumor cells and the histological and clinical features of the disease. However, even for well characterized adult cancers, it is widely accepted that tumorigenesis is a multistage process involving sequential genetic or epigenetic changes. For example, cells that have reentered the cell cycle must become growth factor independent, escape apoptosis, maintain their telomeres, reorganize the surrounding vasculature and acquire invasive properties to become metastatic cancer. Hahn, W. C. & Weinberg, R. A., “Modelling the molecular circuitry of cancer”, Nat Rev Cancer 2: 331-41 (2002).

Because tumor cells change so dramatically over time, there is considerable heterogeneity at both the molecular and histological level. This heterogeneity becomes more complex when one considers that tumor cells from different patients may undergo different genetic changes and the same tumor may be made up of a mixture of cells at different stages of tumorgenesis. For example, approximately 20% of neuroblastomas exhibit an amplification of the MYCN gene but the other 80% are believed to undergo distinct genetic changes. Brodeur, G. M., “Neuroblastoma: biological insights into a clinical enigma”, Nat Rev Cancer 3: 203-16 (2003).

For childhood tumors of the nervous system, the cell of origin and the environment where tumorigenesis occurs are much more difficult to define. This is because many childhood tumors of the nervous system arise from neural progenitor cells which are constantly undergoing changes over the course of development in a highly dynamic environment. Cepko, C. L. et al., “Cell fate determination in the vertebrate retina”, Proc Natl Acad Sci USA 93: 589-95 (1996); Livesey, F. J. & Cepko, C. L., “Vertebrate neural cell-fate determination: lessons from the retina”, Nat Rev Neurosci 2: 109-18 (2001); Dyer, M. A. & Cepko, C. L., “Regulating proliferation during retinal development”, Nat Rev Neurosci 2: 333-42 (2001); Basch, M. L. et al., “Timing and competence of neural crest formation” Dev Neurosci 22: 217-27 (2000).

Unlike most adult tumors, developmental tumors are proliferating in an environment rich in growth factors. Moreover, in the developing nervous system, mitotic and postmitotic cells are migrating in a series of carefully choreographed patterns. Once ,these cells reach their destination, apoptosis precisely trims away a small subset of the excess neurons and glia. Blood vessels are also expanding in the developing nervous system at a rapid rate in patterns directed by the developing neurons and glia. And if proliferation becomes deregulated as a result of genetic and possibly epigenetic changes in the neural progenitor cells, there are several compensatory mechanisms built into progenitor cells to prevent developmental disasters. Efforts to understand each step of tumorigenesis in the context of this changing neural progenitor cell population and dynamic environment presents a tremendous challenge for molecular pathologists seeking to understand the underlying defects in childhood tumors of the nervous system which is a necessary first step to help clinicians target therapies for these malignancies.

A better understanding of the normal developmental processes that regulate formation of the nervous system has proven to contribute to our understanding of tumorigenesis in those tissues and are beginning to help target cancer therapies. Many of the major advances in our understanding of developmental neurobiology have come from the genetic studies in models systems such as mice. For example, a transgenic model of neuroblastoma that recapitulates MYCN amplification in neural crest progenitor cells relied on understanding of normal neural crest development to target ectopic MYCN expression to the appropriate cell at the appropriate time during development. Weiss, W. A., et al., “Targeted expression of MYCN caused neuroblastoma in transgenic mice”, Embo J 16: 2985-2995 (1997). Also, the early work on the role of the hedgehog signaling pathway in cerebellar development has provided a critical link to mutations in this pathway in medulloblastoma. Goodrich, L. V., et al., “Altered neural cell fates and medulloblastoma in mouse patched mutants”, Science 277: 1109-1113 (1997). Similarly, recent advances in our understanding of the role of the Rb family in regulating retinal development has been a major factor in designing in vivo genetic models that faithfully recapitulate human retinoblastoma.

While mouse models have their limitations, when combined with xenograft models using human tumor cell lines and molecular pathology of human cases, they can provide some missing links that may otherwise be overlooked. This example highlights the importance of a new animal model for the study of retinoblastoma.

I. Neural Stem Cells and Neural Progenitor Cells

The cell of origin for many childhood tumors of the central and peripheral nervous system are immature dividing cells. Among these proliferating undifferentiated cells there is experimental evidence to indicate that there are two distinct populations, progenitor cells and stem cells. In both the central and peripheral nervous systems, multipotent dividing progenitor cells undergo progressive rounds of cell division and give rise to different classes of neurons and glia at different stages of development. McConnell, S. K., “Plasticity and commitment in the developing cerebral cortex”, Prog Brain Res 105: 129-143 (1995); Dyer ed.; Livesey supra.; Bronner-Fraser, M., “Molecular analysis of neural crest formation”, J Physiol Paris 96: 308 (2002). Neural crest progenitor cells even have the potential to give rise to non-neuronal cells including cartilage and melanocytes. Garcia-Castro, M. and Bronner-Fraser, M., “Induction and differentiationof the neural crest”, Curr Opin Cell Biol 11: 695-8 (1999); LaBonne, C. and Bronner-Fraser, M., “Molecular mechanisms of neural crest formation” Annu Rev Cell Dev Biol 15: 81-112 (1999).

Neural progenitor cells are believed to undergo unidirectional changes in their competence to give rise to the different cell populations. That is, the potential of an early progenitor cell is different than the potential of a late progenitor cell. At the end of histogenisis, when all cell types have been generated, the last remaining neural progenitor cells exit the cell cycle and undergo terminal differentiation.

While neural progenitor cells share many features of neural stem cells, there are two important distinctions. First, progenitor cells do not retain their potential to make all of the cell types in the tissue of interest. As mentioned above, with each round of cell division, their developmental potential becomes more restricted. Second, they do not retain an unlimited proliferative potential. That is, they eventually undergo terminal cell cycle exit and the differentiated neurons and glia rarely re-enter the cell cycle. Dyer supra; Fischer, A. J. and Reh, T. A., “Muller glia are a potential source of neural regeneration in the postnatal chicken retina”, Nat Neurosci 4: 247-52 (2001).

Stem cells are a specialized subset of progenitor cells that retain the ability to give rise to all the cell types in the tissue, and retain the ability to self -renew by cell division. It is difficult to identify these cells during development because they are so similar to progenitor cells and this has led to much of the confusion in the use of the term “stem cell” to include progenitor cells and stem cells.

Many of the best-characterized stem cells have been studied in fully differentiated tissues because they are much easier to identify when surrounded by postmitotic differentiated cells. It is also important to point out that many of these stem cell populations have only been defined experimentally and as yet have no normal in vivo function. Of course, this is of little consequence when considering the use of these cells for treatment of degenerative disorders.

In childhood cancer of the nervous system, we are concerned primarily with the normal and aberrant function of progenitor cells. This is because they make up the vast majority of cells in the developing nervous system and are likely to be the cells that undergo genetic alterations ultimately giving rise to cancer. It cannot be ruled out that childhood cancer of the nervous system arises from rare stem cells in those tissues, but current estimates of stem cell frequency in the developing neural crest (Kruger, G. M., et al., “Neural crest stem cells persist in the adult gut but undergo changes in self-renewal, neuronal subtype potential, and factor responsiveness”, Nueron 35: 657-69 (2002)), cerebellum (Tamaki, S., et al., “Engraftment of sorted/expended human central nervous system stem cells from fetal brain”, J Neurosci Res 69: 976-86 (2002)) and retina (Tropepe, V., et al., “Retinal stem cells in the adult mammalian eye”, Science 287: 2032-6 (2000)) make such a possibility unlikely. Moreover, neuroblastoma, medulloblastoma and retinoblastoma rarely present in adults who are believed to retain stem cells in each of those neural tissues.

II. Retinoblastoma

Retinal Development. Early during brain development, the optic cup forms as an outcropping of the forebrain. The retinal progenitor cells that make up the optic cup divide and give rise to the seven classes of retinal neurons and glia over the course of development in an evolutionarily conserved birthorder. Importantly, the different cell types are generated from multipotent retinal progenitor cells and it has been proposed that retinal progenitor cells undergo unidirectional changes in competence during development. That is, at a given stage of development, retinal progenitor cells are only competent to give rise to a subset of the postmitotic cell types generated at that developmental stage.

This model implies that there are intrinsic changes in the retinal progneitor cells during development. It has also been demonstrated that extrinsic dues regulate retinal progenitor cell development and thus, it is the balance between intrinsic and extrinsic cues that ultimately dictates the precise generation of the different cell types in the retina. This general scheme, the generation of neural and glial cell types from multipotent progenitor cells in a characteristic order, is common throughout the nervous system. As with other regions of the developing nervous system, there are not only temporal changes in retinal progenitor cells during histogenesis but spatial heterogeneity has been reported as well.

Clinical Features. Retinoblastoma is a childhood tumor of the neural retina that is diagnosed in 95% of cases before 5 years of age and usually within the first year of life. 11% of all cancers in infants (<1 year old) are retinoblastoma, making it the third most common after neuroblastoma and leukemia. In children under the age of 15, retinoblastoma accounts for 3% of all cancers. Each year, there are approximately 300 new cases of retinoblastoma in the United States.

Like medulloblastoma and neuroblastoma, retinoblastoma is believed to arise from a neural retina progenitor cell during development. The evidence for this is severalfold. First, inactivation of the Rb gene (see below) is believed to occur during DNA replication which primarily occurs in dividing retinal progenitor cells during development.

Second, retinal tumors have been found to initiate during fetal development when retinal progenitor cells are actively dividing. Moreover, this tumor never presents in older children or adults suggesting that once the retinal neurons and glia differentiate they are not susceptible to malignant transformation.

Third, molecular analysis of primary retinal tumors has revealed a wide variety of differentiation markers including glial and neuronal markers. Indeed, marker expression for every retinal cell type has been reported in retinoblastoma. If a retinal progenitor cell sustains a mutation in its Rb gene at a particular time during development and initiates the process of becoming a tumor, it is likely that some of the differentiation markers expressed in cells normally made at that time during development would be expressed in the tumor. Considering that some tumors will sustain Rb gene mutations early and others much later during development, it makes sense that different tumors would express different cohorts of differentiation markers. Thus, rather than pointing to a differentiated cell of origin, analysis of differentiation markers in retinoblastomas has lent support to the idea that the cell or origin is a multipotent retinal progenitor.

Metastatic retinoblastoma is among the most deadly childhood tumors. If it metastasizes beyond the eye, the survival rate is only 5-10%. However, if diagnosed early, enucleation can save 90-95% of patients. Some children present with unilateral retinoblastoma with a small number of tumor foci while others present with multifocal bilateral retinoblastoma. These different forms of the clinical presentation (unilateral versus bilateral) reflect the molecular genetics of retinoblastoma gene inactivation (see below). Pediatric screening followed by enucleation is the current approach to unilateral retinoblastoma. Current efforts for treatment are focused on saving vision for bilateral retinoblastoma patients.

Molecular Genetics. As with the other childhood tumors of the nervous system discussed above, there are sporadic and heritable forms of retinoblastoma. However, unlike neuroblastoma and medulloblastoma in which the heritable form is a relatively small proportion of the total number of cases, approximately 40% of children with retinoblastoma exhibit the heritable/bilateral form.

Unilateral and bilateral retinoblastoma result from inactivation of the same gene, RB1. The difference in tumor initiation is believed to reflect the inheritance of the first RB1 mutation. When children inherit a defective copy of the RB1 gene from one of their parents, every cell in their body is heterozygous for that RB1 lesion. All that is required for a retinal tumor to develop is the inactivation of the second RB1 allele during DNA replication in retinal progenitor cells.

In sporadic retinoblastomas, the child does not inherit a defective copy of the RB1 gene. For a tumor to form in the retinae of these children, both copies of RB1 must be inactivated in the same cell. The chance of both RB1 alleles being inactivated by random mutation in a single retinal progenitor cell is much lower than the inactivation of a single RB1 allele in children who have inherited one defective copy of RB1.

While most, if not all, retinoblastomas arise from the inactivation of the RB1 gene, very little is known about the secondary genetic lesions that follow RB1 gene inactivation. The p53 gene has been the most extensively studied and like neuroblastoma and medulloblastoma, there are few mutations in this locus.

Histopathological Correlation. Two histological features of retinoblastoma have been described. Flexner-Wintersteiner and Homer Wright rosettes have been proposed to reflect partially differentiated retinoblastoma cells. Yet, in general, retinoblastoma cells are small undifferentiated cells with high mitotic indices. They form rings of viable cells surrounding the retinal vasculature in the vitreous that has been co-opted form the retinal surface. Depending on the extent of vascularization, there may be necrotic calcified debris in the vitreous from dead tumor cells that were displaced away from the vasculature as the tumor cells divided. Another feature of retinoblastomas is the occurrence of vitreal seeds which are small clusters of tumor cells free floating in the vitreous. These vitreal seeds present one of the major challenges for the treatment of retinoblastoma because they can form new tumor foci after chemotherapy is complete.

For metastatic retinoblastoma, tumor cell invasion usually occurs at the optic nerve. None of these histological features have been correlated with molecular alterations due to the paucity of molecular analysis of the genetic events downstream of RB1 gene inactivation.

Xenograft Mouse Model. There are currently two widely used retinoblastoma cell lines available from the ATCC called Y79 and Weril. These cell lines have been cultured for a significant amount of time and have likely undergone genetic changes since their original isolation. Thus, it will be important to isolate new cell lines from patients and repeat any xenograft experiments with these fresh isolates to verify the data from Y79 and Weril cells.

Until recently, the only xenograft model of retinoblastoma involved injecting Y79 and Weri1 cells into the eyes of adult immunocompromised mice. The intraocular environment of the adult eye is dramatically different than that of the developing eye and thus we sought to develop a xenograft model that more faithfully recapitulated the human disease.

To achieve this, we have injected the Y79 or Weri1 cells into the eyes of newborn rats. Immunosuppresion is not required because at this stage of development the rats are immunonaive and do not reject the human cells. Moreover, the stage is more appropriate for the timing during development in humans when the tumors are likely to form. These xenografted tumor cells (1,000 cells per eye) fill the vitreous within two weeks and exhibit many o the features of the human disease including vascular reorganization, invasion at the optic nerve, and calcification characteristic of cell death in regions of the vitreous where oxygen and nutrients exchange are limited.

To follow their growth and invasion, the engrafted cells are labeled with a GFP reporter gene that is part of a novel Tet-regulatable two plasmid system that allows ectopic expression of genes believed to be involved in tumorigenesis in retinal xenografts. More importantly, we are currently using this xenograft system to study several new chemotherapeutic treatments. While these are not targeted therapies (see below) they may help to improve the rate of vision preservation in bilateral retinoblastoma patients. Efforts are underway to isolate new retinoblastoma cell lines, which should more faithfully recapitulate the genetic alterations that occur in human tumors.

Clonal Inactivation of Rb Family. The orthotopic, developmentally appropriate xenograft model described above is optimal for testing new chemotherapeutic treatment protocols because the site of injection, stage of development, and histological features are remarkably similar to the human disease. However, despite these advantages, xenografts are somewhat artificial in that a homogenous population of cells that are already transformed are placed into a naive environment. In human patients, the developing cancer cells interact with the surrounding tissue to give a heterogenous population of cells and a tumor with spatial heterogeneity. Ideally a high throughput screen involving a genetic model of retinoblastoma that faithfully recapitulated the human disease would be a better system for developing new treatment protocols.

The RB1 gene was the first tumor suppressor gene cloned in humans and the first tumor suppressor knocked out in mice. It was expected that Rb heterozygous mice would phenocopy humans and present with bilateral retinoblastoma. Interestingly, Rb^{±} mice do not develop retinoblastoma. They do present with pituitary tumors indicating that Rb is a tumor suppressor in mice. Subsequent studies revealed that the human RB gene was able to rescue the embryonic lethality of Rb-deficient embryos indicating that the difference between humans and mice is not a reflection of the primary structure of the Rb gene in these two species.

In order to account for the lack of intraocular malignancy in Rb^{±} mice, researcher proposed that there may be redundancy between Rb and the other Rb family members p107 and p130 in the developing retina. To date, this has not been directly verified but there are a few intriguing clues from genetic studies in mice. Chimeric mice made from Rb deficient cells do not develop retinoblastoma showing that Rb gene inactivation alone is not sufficient for tumor formation. Tumors do form when Rb, p107 and p53 are inactivated. It is not known if p107, p53 or both genes must be inactivated for retinoblastoma to form in the mouse retina. Nor is it known if these effects are cell autonomous. A variety of non-cell autonomous effects have been reported for Rb in the mouse so it is possible that some of the contributions of Rb, p107 or p53 are non-cell autonomous.

Several different transgenic mouse models have been developed that express oncogenes such as T antigen from SV40 virus or E1A from adenovirus but these experiments have not significantly advanced our understanding of the genetics of retinoblastoma in the mouse. This is because oncogenes are promiscuous and can bind and inactivate all of the Rb family members as well as other proteins that regulate proliferation and apoptosis. Nor do these mice advance our understanding of cell autonomous and non-cell autonomous contributions of the different genes that may contribute to tumorigenesis in the mouse because the expression pattern of the various transgenes are broad and poorly characterized.

Rather than use transgenic approaches, we have utilized a series of retroviral vectors to induce changes in single retinal progenitor cells in vivo. We start with replication incompetent retroviruses that can only infect dividing retinal progenitor cells. These vectors have an internal ribosome entry site and a reporter gene so we can identify the infected cells. Then, a gene of interest is cloned into the viral vector and a stock is made. The stock is then injected into the eyes of newborn mice or rats and several weeks later the clones of cells that originated from individual infected retinal progenitor cell are analyzed. By diluting the virus, we can achieve a small number of tumor foci (1-5) in each retina.

The advantage of this system is that tumors arise from individual retinal progenitor cells rather than a large population and we can begin to study the cell autonomy of these effects. When combined with the xenograft model these studies provide a complimentary model for drug testing as well as reagents to begin to identify the downstream mutational events that lead from Rb inactivation to tumor formation.

In the first series of experiments we cloned in the E1A 13S cDNA into our retroviral vectors. Following infection of P0 retinal progenitor cells in vivo, clonal hyperplasia was observed. Interestingly, no tumors formed in these animals even after 8 weeks following infection.

As discussed above, there is some data to suggest that for retinoblastoma to form in the mouse, the p53 gene must be inactivated. To test this directly, we injected the same E1A virus in to the eyes of newborn p53 deficient mice. Within three weeks, at an infection rate of 1-5 clones per retina, retinoblastoma formed. This system has the distinct advantage in that tumors arise from a single cell rather than a large pool of progenitors of differentiated neurons as in transgenic models of retinoblastoma.

As mentioned earlier, retinal progenitors are temporally and spatially heterogeneous. It is possible therefore that some retinal progenitors may be more or less susceptible to transformation. To begin to study this question, we have developed a procedure for injecting retroviruses into the eyes of embryonic mice in utero. Previously, it was technically too difficult to perform in vivo viral injections at embryonic stages because few animals survived after birth. Using this approach, we are now testing the susceptibility of early retinal progenitor cells to E1A mediated transformation in different genetic backgrounds.

While E1A is useful for inducing tumors from individual retinal progenitor cells in the developing retina, it is an oncogene that binds to all of the Rb family members as well as other proteins such as p300. To more precisely dissect the molecular pathway leading to retinal tumorigenesis in the developing mouse retina we have developed a retroviral based system to inactivate specific genes in individual retinal progenitor cells. Specifically, we have generated and tested a conditional knockout retrovirus encoding the Cre recombinase. By infecting retinal progenitor cells in mice carrying the LoxP recombination site can selectively inactivate genes in individual retinal progneitor cells in vivo. Conditional knockout mice are now available for both Rb and p53 and by crossing these mice to p107 knockout mice we can attempt to discern precisely which of these genes are important for the generation of retinal tumors in mice in vivo. Moreover, because the individual clones of cells infected with the retroviruses are surrounded by 10,000 to 100,000 uninfected cells, it is reasonable to assume that any changes in proliferation or tumorigenesis are cell autonomous.

Targeted Therapies. There are currently no targeted therapies for retinoblastoma because very little is known about the downstream genetic events that lead to malignant transformation in retinal progenitor cells. The mouse genetic system described herein combined with molecular analyses of primary and cultured human retinal tumors should enable researchers to identify potential candidates for targeted anti-cancer therapy in the treatment of retinoblastoma.

Retinoblastoma Summary. Despite the early discoveries involving the initiation of molecular genetic changes that lead to retinoblastoma, we know very little about the subsequent genetic or epigenetic changes in these tumor cells as they progress from preneoplastic lesion to metastatic cancer. This has been due in part to the lack of a mouse model that faithfully recapitulates the human disease. Transgenic mice ectopically expressing oncogenes in the retina have been of limited use for the study of retinoblastoma because the transgene is expressed in a large number of cells leading to massive hyperproliferation rather than focal transformation.

The recently developed retroviral system that we have used to induce retinoblastoma results in the focal formation of tumors that are much more similar to human retinoblastoma. The limitation of the current retroviral system is that it relies on the E1A oncogene to inactivate the Rb family. A more elegant approach would rely on the inactivation of individual Rb family member in individual retinal progenitor cells using a retrovirus encoding Cre recombinase and the Rb^{Lox} mice. This system is currently being tested.

Recent advances in rodent xenograft models have led to a developmentally appropriate orthotopic xenograft model of retinoblastoma. We are currently using this system to study new chemotherapeutic approaches for the treatment of bilateral retinoblastoma. However, these xenografts rely on retinoblastoma cell lines that were isolated decades ago and efforts are underway to generate new tumor cell lines that have experienced limited time in culture and fewer opportunities for genetic alterations.

Conclusion

The incidence of childhood tumors for retinoblastoma (300 cases per year) accounts for a fairly significant portion of all childhood malignancies. However, retinoblastoma is exceedingly rare compared to adult tumors and not only is it rare, it is difficult to study because of the complexity in the environment. For such a small number of new patients each year, the investment in targeted drug therapy for childhood retinoblastoma is not financially feasible. However, many of the genes and pathways involved in the development of retinoblastoma are found elsewhere in other more prevalent tumor types and children can benefit from targeted therapies for these proteins.

Mouse models play a critical role in such targeting. First, they provide us with many of the basics of the developmental processes of the nervous system that is required for targeting therapy. Second, they provide valuable tools for testing drug therapies including more sophisticated xenograft models as well as genetic models of disease. Third, they provide a system to test compensation and redundancy as well as new genetic lesions that are discovered in human tumors. Even when the genetics in mice are different from the genetics in humans, there may be a common target lesion that could prove quite informative for chemotherapy such as Gli, Beta-catenin MYCN or E2Fs.

Even with the best understanding of the pathways and efficient targeting of the molecule of interest without side effects, there is more to the treatment than initially believed. Specifically, compensation or redundancy may play a critical role.

### Example 2

### Two New Rodent Retinoblastoma Models to Test Combination Chemotherapy

### SUMMARY

Chemotherapy combined with laser therapy and cryotherapy has improved the ocular salvage rate for children with bilateral retinoblastoma. However, children with late-stage disease often experience recurrence shortly after treatment. To improve the vision salvage rate in advanced bilateral retinoblastoma, we have developed and characterized two new rodent models of retinoblastoma for screening chemotherapeutic drug combinations. The first model is an orthotopic xenograft model in which GFP-labeled human retinoblastoma cells are injected into the eyes of newborn rats. This model recapitulates many features of retinal tumorigenesis in humans including the developmental stage of tumor onset. The second model uses a replication-incompetent retrovirus (L1A-E^{E1A}) encoding the E1A oncogene. Clonal, focal tumors arise from mouse retinal progenitor cells when L1A-E^{E1A} is injected into the eyes of newborn p53^{-/-} mice. Using these two models combined with pharmacokinetic studies and cell culture experiments, we have tested the efficacy of topotecan, carboplatin, vincristine, and combinations thereof as chemotherapeutic agents for the treatment of retinoblastoma. The combination of topotecan and carboplatin most effectively halted retinoblastoma progression in our rodent models.

Introduction

Retinoblastoma is a childhood tumor of the retina that arises during development and is usually detected during the first few years of life. In infants (younger than 1 year), retinoblastoma is the third most common form of cancer, after neuroblastoma and leukemia. Both heritable and sporadic forms of retinoblastoma result from inactivation of the retinoblastoma susceptibility gene, RB1. Children who inherit one defective copy of RB1 are likely to develop bilateral, multifocal retinoblastoma as a result of inactivation of the second RB1 allele in retinal progenitor cells during development (DiCiommo, D. et al., “Retinoblastoma: the disease, gene and protein provide critical leads to understand cancer”, Semin Cancer Biol 10: 255-69 (2000)). Sporadic retinoblastoma also results from RB1 inactivation. However, due to the reduced probability of two spontaneous RB1 mutations occurring in the same retinal progenitor cell during development, unilateral retinoblastoma tends to develop in children with the sporadic form of the disease (DiCommo, D. et. al., id; Knudson, A. & Strong, L., “Mutation and cancer: neuroblastoma and pheochromocytoma”, Am. J. Hum. Genet. 24: 514-522 (1972)). Even with current anticancer therapy, metastatic retinoblastoma is fatal in approximately 80% to 90% of cases (Rodriguez-Galindo, C. et al., “Treatment of metastatic retinoblastoma”, Ophthalmology 110: 1237-40 (2003)). To minimize the risk of metastatic disease, patients with unilateral retinoblastoma undergo enucleation. Children with retinoblastoma in both eyes often undergo anticancer therapy to avoid bilateral enucleation and blindness. Chemotherapy combined with laser treatment and cryotherapy has improved the eye salvage rate for children with bilateral retinoblastoma and preserved vision for some patients (Rodriguez-Galindo, C. et al., “Treatment of intraocular retinoblastoma with vincristine and carboplatin”, J Clin Oncol 21: 2019-25 (2003)). However, late-stage bilateral retinoblastoma remains difficult to treat with this approach (Rodriguez-Galindo, C. et al., id).

There are currently two types of rodent models of retinoblastoma—xenograft and transgenic mouse models. The retinoblastoma xenograft model relies on injecting more than 1×10⁶ cultured human retinoblastoma cells into the flank of adult immunocompromised (SCID) mice (del Cerro, M. et al., “Transplantation of Y79 cells into rat eyes: an in vivo model of human retinoblastomas”, Invest Ophthalmol Vis Sci 34: 3336-46 (1993)). This model fails to recapitulate the intraocular environment or developmental milieu that is present in children with retinoblastoma. The existing transgenic mouse models of retinoblastoma rely on the broad ectopic expression of the SV40 T oncogene to lead to massive hyperproliferation (O'Brien, J. M. et al., “A transgenic mouse model for trilateral retinoblastoma”, Arch Ophthalmol 108: 1145-51 (1990)). The limitation of this transgenic mouse model is the lack of focal clonal tumors. Large regions of the retina are genetically altered, predisposing the cells to undergo malignant transformation. However, retinoblastomas arise from one individual cell leading to a small number of tumor foci in children.

We have focused on the developmental environment and focal origins of retinoblastoma in developing two new rodent models for testing the efficacy of combination chemotherapy. In our xenograft model, we injected 1,000 GFP-labeled human retinoblastoma cells into the eyes of newborn rats to create a developmentally appropriate, orthotopic xenograft model of childhood retinoblastoma. In our genetic model, we injected a replication-incompetent retrovirus encoding the E1A oncogene into newborn p53-deficient mice to induce clonal retinal tumors that arise from 1-5 individual foci. The three drugs used in this study, topotecan (TPT), carboplatin (CBP), and vincristine (VCR), have distinct mechanisms of action and have used previously for the treatment of childhood cancer of the nervous system (Thompson, J. et al., “Animal models for studying the action of topoisomerase I targeted drugs”, Biochem Biophys Acta 1400: 301-19 (1998); Thompson, J. et al.,“Synergy of topotecan in combination with vincristine for treatment of pediatric solid tumor xenografts”, Clin Cancer Res 5: 3617-31 (1999); Houghton, J. A. et al., “Childhood rhabdomyosarcoma xenografts: responses to DNA-interacting agents and agents used in current clinical therapy”, Eur J Cancer Clin Oncol 20: 955-60 (1984); Houghton, J. A. et al., “Determinants of intrinsic sensitivity to Vinca alkaloids in xenografts of pediatric rhabdomyosarcomas”, Cancer Res 44: 582-90 (1984); Gaynon, P. S. et al., “Carboplatin in childhood brain tumors. A Children's Cancer Study Group Phase II trial”, Cancer 66: 2465-9 (1990); Allen, J. C. et al., “Carboplatin and recurrent childhood brain tumors”, J Clin Oncol 5: 459-63 (1987)). TPT is a topoisomerase I inhibitor that causes DNA breaks (Jones, S. F. & Burris, H. A., 3rd “Topoisomerase I inhibitors: topotecan and irinotecan”, Cancer Pract 4: 51-3 (1996)); CBP damages DNA by the formation of platinum-DNA adducts(Tonda, M. E. et al., “Formation of platinum-DNA adducts in pediatric patients receiving carboplatin”, Pharmacotherapy 16: 631-7 (1996)); and VCR disrupts microtubules during the M phase of the cell cycle (Stenberg, P. E. et al., “Disruption of microtubules in vivo by vincristine induces large membrane complexes and other cytoplasmic abnormalities in megakaryocytes and platelets of normal rats like those in human and Wistar Furth rat hereditary macrothrombocytopenias”, J Cell Physiol 162: 86-102 (1995)). Our data show that the combination of TPT and CBP is the most effective treatment for retinoblastoma in our animal models. While VCR is an effective anti-tumor drug in culture and it penetrates the blood-ocular barrier, the slow kinetics of action may reduce efficacy in vivo.

Materials and Methods

Cell Culture. Y79 and Weri1 cells were obtained from the American Type Culture Collection (Manassas, Va.) and maintained in culture in RPMI medium with 10% FCS (McFall, R. C. et al., “Characterization of a new continuous cell line derived from a human retinoblastoma”, Cancer Res 37: 1003-10 (1977)). Stable lines expressing GFP were generated by transfecting pTET-1 (M.A.D. unpublished) into Y79 and Weril cells by using TransFast (Promega, Madison, Wis.); clones were isolated in the presence of hygromycin. To measure live cells, 0.5 ug/ml calcein was used; to measure dead cells, 4 uM ethidium bromide was used.

Immunolabeling and TUNEL and BrdU Assays. Y79 and Weri1 retinoblastoma cells were immunolabeled as described previously for dissociated mouse retinae (Dyer, M. A. & Cepko, C. L., “p27Kip1 and p57Kip2 regulate proliferation in distinct retinal progenitor cell population”, J. of Neurosci 21: 4259-71 (2001); Zhu, C. C. et al., “Six2-mediated auto repression and eye development requires its interaction with members of the Groucho-related family of co-repressors”, Development 129: 2835-49 (2002); Dyer, M. A. & Cepko, C. L., “p57(Kip2) regulates progenitor cell proliferationand amacrine intemeuron development in the mouse retina”, Development 127: 3593-605 (2000)). For in vivo studies, BrdU (100 mg/kg body weight) was administered via tail vein injection. BrdU and TUNEL assays were carried out as described previously (Dyer, M. A. et. al., id, Zhu, C. C. et. al., id; Dyer, M. A. et. al., id). Detailed protocols are available at http://www.stjude.org/dyer.

Fluorescent-Activated Cell Sorting. DNA content was analyzed by dissociating tumors with trypsin (Dyer, M. A. et. al., id; Zhu, C. C. et. al., id; Dyer, M. A. et. al., id) and sorting cells by fluorescent-activated cell sorting (FACS). Cells were washed with PBS, and resuspended in a solution containing 0.05 mg/ml propidium iodide, 0.1% sodium citrate and, 0.1% Triton-X100. Samples were then treated with RNase, filtered through a 40-um nylon mesh, and analyzed on a FACScan (Beckton-Dickson, Franklin Lakes, N.J.).

Microarray Hybridization. The H1 human cDNA microarray was printed at the Hartwell Center for Bioinformatics & Biotechnology at St. Jude Children's Research Hospital. The gene list is available at http://www.ncbi.nlm.nih.gov/geo/ (GEO Accession: GLP345). RNA was isolated from approximately 20×10⁶ cells in triplicate for each treatment using Trizol (Invitrogen, Carlsbad, Calif.), and 10 ug total RNA underwent one round of linear amplification using the RiboAmp System (Arcturus Applied Genomics, Carlsbad, Calif.) to yield 100 ug of RNA. The RNA was indirectly labeled using aa-dUTP and conjugated with Cy3 and Cy5. Data were then filtered (see http://www.ncbi.nlm.nih.gov/geo/; GLP345 for details), and cluster analysis was performed.

Pharmacology. Two-week-old rats were treated with TPT (Hycamtin; GlaxoSmithKline, Research Triangle Park, N.C.) at a maximum tolerated dose (MTD) of 2 mg/kg body weight, CBP (Paraplatin; Bristol-Myers Squibb, New York, N.Y.) at an MTD of 70 mg/kg, and VCR (Mayne Pharma (USA Inc., Paramus, N.J.) at an MTD of 0.5 mg/kg; all agents were administered via tail vein injection. TPT was measured by HPLC UV absorption as described (Thompson, J. et. al., id). Atomic absorption spectrometry of ultrafiltered and nonfiltered samples was used to measure platinum levels (Simpson, A. E. et al., “Transscleral diffusion of carboplatin: an in vitro and in vivo study”, Arch Ophthalmol 120: 1069-74 (2002)). Liquid scintillation was used to measure levels of [H³]-VCR (70-90 Ci/mmol; PerkinElmer, Wellesley, Mass.). Mice received TPT, CBP, and VCR at a dose and schedule similar to that used to treat children with retinoblastoma (Schouten-Van Meeteren, A. Y. et al., “Overview: chemotherapy for retinoblastoma: an expanding area of clinical research”, Med Pediatr Oncol 38: 428-38 (2002)) or other brain tumors.

Animals, Tissues, and Retroviruses. The p53^{-/-} mice were obtained from the National Cancer Institute. All mice were crossed to C57B1/6 mice purchased from Charles River Laboratories (Wilmington, Mass.). Timed-pregnant Sprague-Dawley rats were also purchased from Charles River Laboratories. Human fetal retinal tissue was obtained from Advanced Biosciences Resources, Inc. (Alameda, Calif.). Retroviral procedures have been described elsewhere (Dyer, M. A. & Cepko, C. L. id.; Dyer, M. A., “Regulation of proliferation, cell fate specification and differentiation by the homeodomain proteins Prox1, Six3, and Chx10 in the developing retina”, Cell Cycle 2: 350-7 (2003); Dyer, M. A. & Cepko, C. L., “The p57Kip2 cyclin kinase inhibitor is expressed by a restricted set of amacrine cells in the rodent retina”, J Comp Neurol 429: 601-14 (2001); Zhang, J. et al., “Rb regulates proliferation and rod photoreceptor development in the mouse retina”, Nat Genet 36(4): 351-60 (April 2004)).

Real-Time RT-PCR. Real-time PCR experiments were performed using the ABI 7900 HT Sequence Detection System (Applied Biosystems, Foster City, Calif.). Primers and probes were designed using Primer Express software (ABI). Probes were synthesized with 5′-FAM and 3′-BHQ. RNA was prepared using Trizol, and cDNA was synthesized using the Superscript system (Invitrogen). Samples were analyzed in duplicate and normalized to GAPDH expression levels.

Microscopy and Tumor Reconstruction. Brightfield and single-cell fluorescent images were obtained using a Zeiss axioplan 2 fluorescent microscope with the Zeiss AxioCam digital camera. Fluorescent images of tissue sections were obtained using a Leica TCSNT confocal microscope. Tumor reconstruction was carried out using BioQuant 5.0 software.

Viable Cell Calculation Following Drug Treatment. Following drug treatment the total number of cells (N) were scored and this was followed by calcein (C) and EthBr (E) staining to determine the proportion (C/C+E) of those cells that were metabolically active (M). The proportion of cells that had initiated apoptosis (T) was determined by a TUNEL assay. In a separate experiment we determined that 90.7% of TUNEL labeled cells were also identified as non-metabolically active by EthBr staining. Therefore, 1-0.907(T) is the proportion of non-viable cells that had initiated apoptosis but were not yet detected by other means. All of these data were combined to obtain the number of viable cells in the following equation: number of viable cells =N×M×(1-0.907(T)) The proportion of viable cells is the ratio of the number of viable cells for the treated sample over the untreated sample.

Results

Gene Expression in Retinoblastoma Cell Lines Resembles Primary Tumors.

The two retinoblastoma cell lines (Y79 and Weril) that have been most widely used for testing anti-tumor therapies have been cultured for many years. To determine if these cells have sustained genetic or epigenetic alterations that result in dramatically different proliferation or apoptosis properties, we analyzed the expression of 15 genes that regulate proliferation and apoptosis in Y79 and Weri1 cells and compared those data to two cell lines maintained in culture for a brief period (Rb118 and Rb130) (Griegel, S. et al., “In vitro differentiation of human retinoblastoma cells into neuronal phenotypes”, Differentiation 45: 250-7 (1990); Griegel, S. et al., “Newly established human retinoblastoma cell lines exhibit an “immortalized” but not an invasive phenotype in vitro”, Int J Cancer 46: 125-32 (1990)) and 30 primary tumors from cases of bilateral and unilateral disease. Real-time RT-PCR was used to quantitate mRNA expression levels and immunolabeling was used to analyze protein expression. We found that the genes that were expressed in cultured retinoblastoma cell lines were also expressed in primary tumors.

In a subsequent experiment, we compared the expression of cell cycle and apoptosis genes in retinoblastoma cells to human fetal retinal progenitor cells which is the cell of origin for retinoblastoma (see Example 1 for a discussion). The levels of cell cycle regulatory gene expression were similar in retinal progenitor cells and retinoblastoma, except for the expression of genes that encode the Rb family proteins. During normal retinal development in humans, RB1 is the key regulator of proliferation (Gray et al., submitted). However, in the tumor samples, p107 and p130 were upregulated to high levels. Moreover, several genes that mediate apoptosis or stress response such as p53, p21, Bcl-X7, and p14ARF were expressed at higher levels in the tumors than in normal retinal progenitor cells.

Orthotopic Retinoblastoma Xenograft in the Developing Eye Recapitulates Human Disease. Having demonstrated that the well-characterized retinoblastoma cell lines (Y79 and Weri1) express a similar cohort of cell cycle and apoptosis genes as primary tumors, we developed an orthotopic retinoblastoma xenograft model of retinoblastoma using Y79 cells. Specifically, we transplanted 1,000 cells into the vitreous of newborn rats to approximate the stage of tumor formation in humans (Clancy, B. et al., “Translating developmental time across mammalian species”, Neuroscience 105: 7-17 (2001)). Immunosuppression was not required in these animals, because rats are immunonaive for the first 24 hours after birth. For some experiments, the retinoblastoma cells were labeled with a GFP transgene to unambiguously establish tumor boundaries. Within 2 weeks, the engrafted cells proliferated and filled the vitreous. They also reorganized the retinal vasculature and invaded the optic nerve.

To establish a baseline prior to chemotherapy treatment in our retinoblastoma xenograft model, we analyzed cell number, survival, proliferation, apoptosis, and gene expression 2 weeks after engraftment. One hour before analysis, the rats received an injection of BrdU to label the cells in the S-phase of the cell cycle, a measure of the fraction of dividing cells. The engrafted cells were microdissected from the normal tissue and dissociated; the total cell number was then scored. Calcein and ethidium bromide were used to measure metabolically active cells and dead cells, respectively. Approximately 85-95% of Y79 cells were metabolically active after two weeks in the intraocular environment. The fraction of dividing cells was estimated by staining with an anti-BrdU antibody and scoring the fraction of immunopositive cells. 8-10% of Y79 tumor cells incorporated BrdU. To estimate the proportion of cells that had initiated apoptosis, we performed a TUNEL assay. 2-3% of cells had initiated apoptosis using the TUNEL assay. FACS analysis demonstrated that 52.6% were in G0/G1, 40.2% in S and 7.2% in G2/M. The ratio of tumor to retinal volume (0.47±0.21) was determined by removing the eye and reconstructing serial sections.

Response of Cultured Retinoblastoma Cells to Chemotherapeutic Drugs. To determine whether TPT, CBP, or VCR is an effective treatment of retinoblastoma, we tested their individual effects on Y79 and Weril retinoblastoma cells; we examined cell survival, proliferation, apoptosis, gene expression, and cell cycle arrest. For all of the cell culture studies, retinoblastoma cells were exposed to drugs at a cell density corresponding to their optimal proliferation and cell survival. The cell number, proportion of metabolically active cells, and proportion of apoptotic cells were combined to generate the proportion of viable cells (see Materials and Methods for calculations). The proportion of dividing cells was estimated by labeling the cultures with BrdU for 1 hour. Initially, cells were exposed to TPT, CBP, or VCR for 8 hours at concentrations ranging from 1 nM to 200 uM, and the cultures were assayed 3 days later. The LD₅₀ of VCR was 5 nM for Y79 cells and 3 nM for Weril cells; the LD₅₀ of TPT was 30 nM for Y79 cells and 19 nM for Weril cells; and the LD₅₀ of CBP was 4 uM for Y79 cells and 5 uM for Weri1 cells.

To determine which combination of chemotherapeutic drugs may be most effective, we treated Y79 and Weril cells with the LD₅₀ of TPT, VCR and CBP for 8 hours. Following treatment, RNA was isolated and microarray hybridizations were carried out using the human H1 cDNA microarray (see Materials and Methods). We found that treatment of retinoblastoma cells with VCR or CBP resulted in similar cellular responses whereas treatment with TPT resulted in distinct cellular responses. Thus, we reasoned that the combination of TPT with VCR or TPT with CBP may be the most effective combination treatments for retinoblastoma. Using the LD₅₀ of TPT, we tested the combination of TPT and CBP and that of TPT and VCR. At concentrations of 2 nM to 200 μM CBP, the TPT and CBP combination more effectively reduced cell viability and proliferation than either drug alone. This effect was additive. Similarly, at concentrations of 1 to 10 nM VCR, the TPT and VCR combination was more effective at reducing cell viability and proliferation than either drug alone. Concentrations of VCR higher than 10 nM showed no added benefit in combination treatment.

In these preliminary studies, we included an 8-h drug exposure, because that is the maximum period of drug exposure for children receiving chemotherapy. However, the time that tumor cells are exposed in situ is probably considerably shorter, so we performed a time course experiment to determine the minimum duration of exposure required for the full effect on retinoblastoma cell survival and proliferation. As little as 15 min of exposure to TPT or CBP was sufficient to achieve the maximum reduction in cell survival and proliferation. The effects of VCR were much slower with a maximum decrease in viability at 4 hours. FACS analysis of these samples revealed that the effects on cell cycle arrest were consistent with the effects on proliferation. For example, retinoblastoma cells exposed to TPT for 10 min arrested in G2 phase in a similar proportion (36%) as those exposed to TPT for 8 h (39%).

Ability to Cross the Blood-Ocular Barrier. One of the primary hurdles for chemotherapeutic treatment of retinoblastoma is drug penetration through the blood-ocular barrier, which is made up of the zonulae occludens, or tight junctions of the pigment epithelium and retinal capillaries into the vitreous (Peyman, G. A. & Schulman, J. A., “Intravitreal drug therapy”, Jpn J Ophthalmol 33: 392-404 (1989)). This is of particular importance, because vitreal seeds represent a primary clinical feature of late-stage bilateral retinoblastoma, and these small clusters of cells cannot be treated effectively with laser therapy or cryotherapy (Rodriquiz-Galindo, C. et. al., id).

To measure retinal and vitreal penetration of TPT, CBP, and VCR, we injected each drug intravenously into 2-wk-old rats; this age corresponded to the stage of treatment in our xenograft model. Drug levels were measured in the vitreous, retina, and plasma at 3 min, 30 min, 1.5 h, 4 h, and 6 h after injection. A substantial amount of TPT penetrated the vitreous, as indicated by the area under the curve (AUC) vitreous/plasma ratio of 0.38 and retina/plasma ratio of 0.7. CBP also had good vitreal penetration (AUC vitreous/plasma=0.59), but only a low level of CBP was present in the retina (AUC retina/plasma=0.03). VCR exhibited the best vitreal and retinal penetration (AUC vitreous/plasma=1.1; retina/plasma 1.0).

Response of Retinoblastoma Xenografts to Chemotherapeutic Drugs. To test the hypothesis that the combination of TPT and CBP is the most effective treatment for late-stage retinoblastoma, we treated 2-wk-old rats that received an intraocular injection of retinoblastoma cells at birth with TPT, CBP, VCR, or the combinations of TPT and CBP or TPT and VCR (16 animals/group). After one course of treatment, the transplanted retinoblastoma cells were analyzed for viability and proliferation as described above. CBP was the most effective single drug in this assay reducing the cell number/eye from 2.4±0.6×10⁶ to 0.79±0.29×10⁶ (p<0.001). TPT combined with CBP was the most potent combination therapy reducing cell number further to 0.22±0.1×10⁶ (p<0.001). FACS analysis and microarray hybridization demonstrated that these drugs were having the same effect on gene expression and cell cycle arrest in an intraocular environment as in culture. VCR showed little effect on retinoblastoma cell survival in our xenograft model using this short-term assay. 3-dimensional reconstruction of the tumor and retina confirmed that the tumor volume was reduced following chemotherapy treatment.

We were surprised that VCR was not effective at halting retinoblastoma growth in the orthotopic xenograft model because VCR is often combined with other chemotherapeutic agents in the treatment of retinoblastoma in children. It was possible that there was a minor effect of VCR treatment that was masked in our system by the active proliferation and expansion of the xenograft. One of the hallmarks of VCR exposure is a nucleus with twice the genomic DNA content, because VCR blocks cytokinesis without halting cell cycle progression. To test if there was any VCR-mediated block of mitosis, we scored the proportion of large cells with twice the genomic DNA content in cultured cells, xenografts, and tumors removed from patients following VCR treatment. Approximately 98% of cells treated with 5 nM VCR for 8 h exhibited twice their normal DNA content; 2%±1% of xenografts and 5%±1.5% of human tumors treated with VCR exhibited morphologic features consistent with continued cell cycle progression without cytokinesis. The proportion of enlarged nuclei in xenografts and primary human tumors following VCR treatment was similar to control xenografts (3%±2%) and primary tumors (4%±1%), respectively.

Response of Focal Mouse Retinal Tumors to Chemotherapeutic Drugs. Having demonstrated that the combination of TPT and CBP was the most effective short-term treatment of retinoblastoma xenografts, we then tested the efficacy of each drug and drug combination by treating mice that had a small number of focal, clonal retinal tumors. We injected the LIA-E^{E1A} retrovirus encoding the 13S E1A oncogene and an alkaline phosphatase (AP) reporter gene into the subretinal space of newborn p53^{-/-} mice at a titer sufficient to achieve one to five clones per retina (Zhang, J. et. al., id). Within 3 wk, infected retinal progenitor cells proliferated, spread laterally through the retinal vasculature, and eventually filled the entire ocular compartment. Newborn p53^{-/-} mice were injected with the LIA-E^{E1A} retrovirus and at 6 weeks of age they received chemotherapeutic treatment (TPT, CBP, VCR, TPT+VCR and TPT+CBP) for 8 weeks in groups of 14-16 animals. Following treatment, retinae were removed, dissociated, stained for AP expression, and the proportion of tumor cells was scored. The dose and schedule of treatment were consistent with that used for the treatment of children with retinoblastoma or other brain tumors. As in the xenograft studies, the combination of TPT and CBP was more effective than either drug alone. As with the xenograft studies, no additional antitumor effect was seen by combining TPT with VCR.

Discussion

We have developed two new rodent models of retinoblastoma that emphasize the initiation of disease during development and focal, clonal tumor formation. One model involves the transplantation of human retinoblastoma cells into the eyes of newborn rats to recapitulate the stage of tumorigenesis in humans and alleviate the need for immunosuppression, as in flank xenografts transplanted into adult animals (Tosetti, F. et al., “N-(4-hydroxyphemyl) retinamide inhibits retinoblastoma growth through reactive oxygen species-mediated cell death”, Mol Pharmacol 63: 565-73 (2003); Howard, M. A. et al., “Effect of butyrate and corticosteroids on retinoblastoma in vitro and in vivo”, Invest Ophthalmol Vis Sci 32: 1711-3 (1991)). The second model relies on the ectopic expression of E1A in individual retinal progenitor cells in the eyes of newbomp53^{-/-} mice resulting in clonal focal retinoblastoma. Both models were used to test TPT, CBP, and VCR individually and the combination of TPT with CBP or VCR for the treatment of retinoblastoma. All three drugs penetrated the retina and vitreous of 2-week-old rats. As predicted by the pharmacokinetic and cell culture data, we found that CBP and TPT were the most effective single drugs for the treatment of retinoblastoma in vivo and that TPT and CBP was the most effective combination.

Retinoblastoma tumor cell lines derived from primary tumors can undergo genetic or epigenetic changes in culture that distinguish them from the original tumor. Many of the differences between cell lines and primary retinoblastoma that have been reported previously were differentiation markers or epitopes of unknown significance. See Griegel, S. et al., “Newly established human retinoblastoma cell lines exhibit an “immortalized” but not an invasive phenotype in vitro“, Int J Cancer 46, 125-32. (1990); DiCiommo, D. et al., “Retinoblastoma: the disease, gene and protein provide critical leads to understand cancer”, Semin Cancer Biol 10: 255-69 (2000); and Griegel, S. et al., “In vitro differentiation of human retinoblastoma cells into neuronal phenotypes”, Differentiation 45: 250-257. (1990). We have found that several genes that regulate proliferation and apoptosis in tumor cells are expressed in both early-passage cell lines (Rb118 and Rb130) and well-established cell lines Y79 and Weri1. As expected, there was some variance in the relative levels of expression between the early passage cell lines, the late passage cell lines and the primary tumors, which may reflect their different proliferation and/or cell death rates. While future studies will benefit from the use of transplanted primary tumors or pools of cells that have undergone limited passage in culture, the use of the extensively characterized Y79 and Weri1 cell lines was appropriate at this time.

Most animal models have inherent strengths and weaknesses and thus we have sought to combine two complementary models of retinoblastoma to gain the most complete data on chemotherapeutic treatment efficacy. We have sought to improve upon the existing xenograft and genetic models of retinoblastoma to screen chemotherapeutic drugs and improve the eye salvage rate of children with late-stage bilateral retinoblastoma. By injecting a small number of human retinoblastoma cells (1,000 cells) into the vitreous of newborn rats, we overcame the need to use immunocompromised animals. In addition, this orthotopic model more faithfully recapitulates the human disease, because the transplanted cells grow in a developmental environment similar to that of the human retinal tumors. The developmental stage is important, because the differences between the fetal eye and adult eye in terms of the growth factors that are expressed, retinal vasculature, and cytoarchitecture may affect retinoblastoma formation. Our genetic model of retinoblastoma also relies on the appropriate developmental stage of tumor initiation. Importantly, by injecting LIA-E^{E1A} into the eyes of newborn p₅₃^{-/-} mice we have created focal, clonal retinoblastoma. The combination of xenograft and genetic models with pharmacokinetic and cell culture studies have provided us with the best picture to date of the efficacy of individual chemotherapeutic treatments.

Not only does combining xenograft and genetic models give us the most information about potential treatment efficacy in vivo, but by using multiple molecular and cellular assays to study drug effect, we can gain additional insights into the best drugs and drug combinations for retinoblastoma treatment. For example, we counted the total number of cells present after exposure to chemotherapeutic drugs, and then we scored the fraction that was still alive as measured by metabolic activity (calcein staining). We then determined whether any calcein positive cells had initiated apoptosis, if they were still proliferating, and what fraction had undergone cell cycle arrest. This analysis was important, because even if a drug does not immediately kill retinoblastoma cells, it could lead to apoptosis or irreversible cell cycle arrest; thus, it may prove to be an effective drug in patients.

Microarray analysis provided even more information about the response of retinoblastoma cells to chemotherapeutic drugs. For example, we found that the p53 pathway was activated after TPT treatment, which may explain why retinoblastoma cells exposed to TPT undergo G2 arrest. CBP, another DNA damaging agent (Tonda, M. E. et.al., id), caused very different changes in gene expression; p53 was not activated and cells arrested in either the G1 or G2 phase. VCR treatment also altered the expression of a different cohort of genes in comparison to TPT treatment. Not only do these molecular analyses indicate that each drug has very different effects on retinoblastoma cells, but also they provide clues about which pathways are active in these cells (e.g., the p53 pathway). These findings may be useful for future studies aimed at developing targeted retinoblastoma chemotherapy.

Vitreal tumor seeds are one of the biggest clinical challenges of treating late-stage bilateral retinoblastoma. These small clusters of 50 to 1,000 cells are a challenge, because individual seeds can settle near the retinal vasculature, begin to divide, and form new tumor foci after treatment. While in the vitreous, seeds are difficult to treat with laser therapy or cryoablation. Therefore, it is essential to test the penetration of potential chemotherapeutic agents into the vitreous.

Vincristine most effectively killed retinoblastoma cells in culture and penetrated the blood-ocular barrier; however, VCR required a relatively long exposure time (4 h) to kill retinoblastoma cells in culture. Thus, it is not surprising that VCR was the least effective at slowing tumor progression in our animal models in vivo. In contrast, TPT and CBP act very quickly (10-30 min) and have good vitreal penetration. Although TPT and CBP were less potent in culture than VCR, their pharmacokinetic parameters and rapid action suggested that they are better drugs for treatment in vivo. Therefore, on the basis of our studies, the combination of TPT and CBP is a promising combination for the treatment of late-stage bilateral retinoblastoma.

Future Directions

One of the major limitations of the drugs and drug combinations that we tested here is that they are general chemotherapeutic agents with some secondary toxicity such as myelosuppression (Tubergen, D. G. et al., “Phase I trial and pharmacokinetic (PK) and pharmacodynamics (PD) study of toptecan using a five-day course in children with refractory solid tumors: a pediatric oncology group study”, J Pediatr Hematol Oncol 18: 352-61 (1996)) that limit the maximum dose that can be administered (Shields, C. L. et al., “Chemoreductin plus focal therapy for retinoblastoma: factors predictive of need for treatment with external beam radiotherapy or enucleation”, Am J Ophthalmol 133: 657-64 (2002); Shields, C. L. et al., “Combined chemoreduction and adjuvant treatment for intraocular retinoblastoma”, Ophthalmology 104: 2101-11 (1997)). Indeed, secondary toxicity is one reason we have not yet tested the triple combination of TPT, CBP, and VCR. One way to minimize such toxicity is to adjust the dose, schedule, and site of administration of the drugs. A recent study using a transgenic mouse model of retinoblastoma found that subconjunctival CBP administration was effective at reducing tumor size (Hayden, B. H. et al., “Subconjunctival corboplatin in retinoblastoma: impact of tumor burden and dose schedule”, Arch Ophthalmol 118: 1549-54 (2000)). Another study of patients with retinoblastoma found that low-dose, frequent administration of TPT more effectively reduced the tumor burden than did infrequent, high-dose administration of TPT (Tubergen, D. G. et. al., id). Therefore, it will be interesting to test the combination of frequent, low-dose TPT administered with weekly doses of VCR and subconjunctival CBP. This regimen may reduce secondary toxicity while retaining effective vitreal and retinal penetration.

Various publications, patent applications and patents are cited herein, the disclosures of which are incorporated by reference in their entireties.

1. A method for preparing an animal model for retinoblastoma comprising introducing cells capable of giving rise to a retinoblastoma into the eye of a host animal in an amount sufficient to cause the growth of a tumor at a time when said animal is immunologically naive.

2. The method of claim 1 wherein said host animal is selected from the group consisting of a monkey, a rabbit, a mouse and a rat.

3. The method of claim 2 wherein said host animal is a rat.

4. The method of claim 1 wherein said host animal has a genetic susceptibility to retinoblastoma formation.

5. The method of claim 4 wherein said host animal has a defect in the p53 gene.

6. The method of claim 1 wherein said cells are selected from the group consisting of retinoblastoma cells, retinal progenitor cells and retinal stem cells.

7. The method of claim 1 wherein said cells are from an established cell line.

8. The method of claim 1 wherein said cells are derived from human retinoblastoma tissue.

9. The method of claim 1 wherein said cells are derived from a human having a genetic susceptibility to retinoblastoma.

10. The method of claim 9 wherein said genetic susceptibility is selected from the group consisting of a deficiency in Rb gene expression, a deficiency in p107 gene expression and a deficiency in p130 gene expression.

11. The method of claim 1 wherein said sufficient amount comprises from about 50 to about 10,000 cells.

12. The method of claim 11 wherein said sufficient amount comprises from about 500 to about 1000 cells.

13. An animal model for retinoblastoma prepared by the method of claim 1.

14. A method for preparing an animal model for retinoblastoma comprising introducing a vector that expresses an oncogene in transfected cells into at least one eye of a host animal in an amount sufficient to cause the proliferation of a cell mass in transfected cells that mimics the early stages of retinoblastoma formation.

15. The method of claim 14 wherein said host animal has no genetic susceptibility to retinoblastoma formation.

16. The method of claim 14 wherein said host animal has a genetic susceptibility to retinoblastoma formation.

17. The method of claim 16 wherein said host animal has a defect in the p53 gene.

18. The method of claim 14 wherein said oncogene is a viral oncogene.

19. The method of claim 18 wherein said viral oncogene is selected from the group consisiting of E1A, E6, E7 and Tag.

20. The method of claim 19 wherein said oncogene is E1A.

21. The method of claim 14 wherein said oncogene is a cellular oncogene.

22. The method of claim 21 wherein said cellular oncogene is selected from the group consisting of Ras, Myc, Abl and Erk.

23. The method of claim 14 wherein the amount of vector used is sufficient to cause the generation of 1-5 clonal retinoblastoma tumors in the eye of said host animal.

24. The method of claim 14 wherein said host animal is selected from the group consisting of a monkey, a rabbit, a mouse and a rat.

25. The method of claim 24 wherein said host animal is a mouse.

26. The method of claim 14 wherein said vector is a retroviral vector.

27. The method of claim 26 wherein said retroviral vector is replication incompetent.

28. An animal model for retinoblastoma prepared by the method of claim 14.
